(19) United States
(12) Patent Application Publication
(10) Pub. No.: US 2009/0062706 A1
(43) Pub. Date: Mar. 5, 2009

(54) Method and apparatus for correcting head and shoulder posture

(76) Inventor: Archie Lee Phillips, Fort Worth, TX (US)

Correspondence Address: MARVIN J. POWELL, 621 Millers Hill, Kennett Square, PA, 19348-2431, US

(21) Appl. No.: 12/228,041

(22) Filed: Aug. 9, 2008

Related U.S. Application Data
(60) Provisional application No. 60/964,279, filed on Aug. 10, 2007.

(51) Int. Cl. A61F 5/00; A61B 19/00

(52) U.S. Cl. 602/19; 128/898

(57) ABSTRACT

The apparatus for correcting head and shoulder posture was invented to help those individuals with the dysfunction of habitually carrying their heads and shoulders in an abnormally extended forward or off balance position.

The role of the apparatus is to assist the dysfunctional components of the neuromuscular system that are responsible for maintaining proper head and should posture as one carries out normal activities such as sitting, standing, walking and running.

Many of society's shoulder, neck and back specifically upper back pain as well as resulting collateral pain in other areas can be attributed to poor head and shoulder posture.

Muscles that control head and shoulder movements can become hyper extended in a forwardly position. Continually carrying the head and shoulders in this position can often become habitual and soft tissues affected can become dysfunctional of varied degrees.

The brain controls all body movements both voluntary and involuntary, voluntary as in moving your arm; involuntary as in breathing. However in dysfunctional muscle tissue the brain signals and/or commands to facilitate movement can be ignored in dysfunctional muscles and no movement may occur. In the case of poor head and shoulder posture, the head and shoulders remain in a sustained extended forward position, nonresponsive to brain impulses.

This apparatus was designed to intervene and assist dysfunctional muscles in responding to brain commands to counter balance the head and shoulders before they become hyper extended for sustained periods if dysfunctional. The apparatus' action is to exhaust hyper extended muscles causing more blood to enter those muscles triggering muscle movement counterbalancing the head and shoulders to the proper position conducive to the activities the body is engaged in at the time. This apparatus facilitates renewed communication between the brain and soft tissues so that normal commands and responses can occur in time, unassisted by the apparatus as one's dysfunction diminishes.

This application claims priority from provisional application Ser. No. 60/964,279, filed Aug. 10, 2007, the entire disclosures of which are hereby incorporated by reference.

### BACKGROUND

Poor head posture can contribute too many of society's aches and pains. Individuals that unconsciously carry their heads and shoulders in an over extended forwardly position often have discomfort in these areas. Many people in our society unconsciously hold their heads in this position while others may recognize this habit but are unable to correct it. The muscles typically affected by this dysfunction are the back of the head, neck, upper back, chest and shoulders. Poor head and shoulder posture can affect other muscles and systems of the body in a negative way as well.

This apparatus aids in the reversal of the forward tilted head and shoulder dysfunction by artificial mechanical stimulation by exerting tension on dysfunctional soft tissue causing the soft tissue to exhaust the tissues' remaining energy thus prompting the body to spontaneously rush oxygenated blood to those soft tissues, promoting the correct soft tissue responses in regards to facilitating proper head and should posture this action allows week tissue to gain strength, hyper extended tissue to rest, and inflamed tissue to heal. The apparatus can be discontinued once the neuromuscular system branches that were dysfunctional become active, communication within the systems normalize and soft tissues become healthy and functional responding to one's normal activities such as sitting, standing, walking or running.

### SUMMARY OF THE INVENTION

The apparatus for correcting poor head and should posture (specifically individuals that carry their head and shoulders in the forward position) assist neuromuscular coordination by artificial mechanical stimulation of dysfunctional muscle tissue, prompting revitalization and/or re-energizing of lazy, fatigued soft tissues.

The common dysfunctional posture positions discussed here occur during normal activities such as sitting, standing, walking and running. When the head and shoulders hyperextend forward and remain in this position for extended periods, gravity on the head creates even more strain on the already tired and lazy muscles to such an extent that the brain's command signals are not effective in getting desired muscle responses in order to correct one's head posture.

This apparatus places additional tension on dysfunctional muscles and sending them to exhaustion. The brain senses this and signals for a rush of nutrients to those muscles producing contracting (muscle movement) to bring the head and shoulder into a corrective and/or balanced posture position. This method of reprogramming dysfunctional soft tissue can be used to aid in correcting or preventing an occurrence of dysfunctional soft tissue problems.

### BRIEF DESCRIPTION OF THE DRAWINGS

FIG. 1 illustrates the physical appearance of the overall apparatus, its components and their locations on the apparatus.

FIG. 2 illustrates an enlarged view of a loop clevis fastener and identifies the individual parts and their functions as part of the size adjustment loops.

FIG. 3 illustrates an enlarged view of a fastener and its individual parts and their functions when connecting the head band to be worn and connecting the tensioning strap to a waist band.

FIG. 4 illustrates the proper head and shoulder position for good posture, balance, and dysfunctional poor positions of individuals that carry their head in an extended forward posture position.

FIG. 5 illustrates how the apparatus is worn for corrective head and shoulder posture therapy.

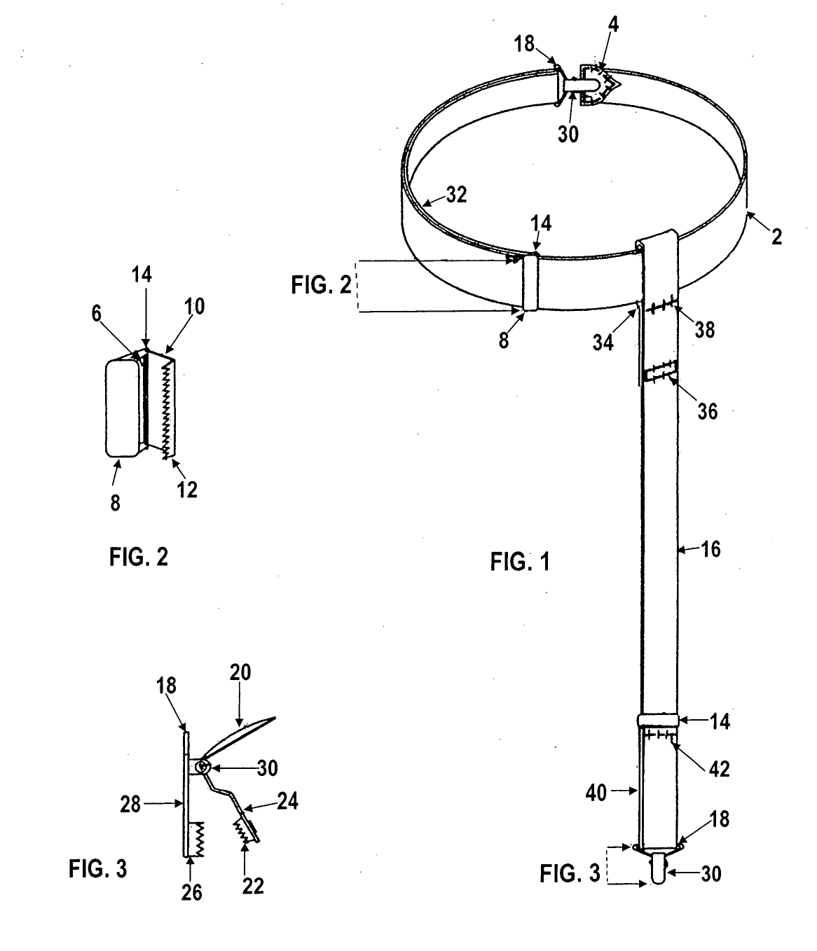

### DETAILED DESCRIPTION OF THE INVENTION

The apparatus consists of an adjustable head band strap. 2. An attached adjustable tensioning strap. 16. The head band strap is made from woven rubber and synthetic cloth material that is elastic when stretched. The head band is 26 inches long×1⅛ inches wide× 1/16 INCHES THICK. The free end 4 of the strap is folded and sew stitched. The strap's opposite free end is threaded thru the loop clevis slot 6 FIG. 2 shows the clevis in the opened (i.e. unlocked position). The loop clevis has a movable fastening tang 8 and a movable retainer 10 which has a saw toothed seat 12. This unit is held together with a hinge pin. The head band strap's free end is then threaded thru the head band fastener loop 18. The fastener FIG. 3 in the opened position has a hinged finger clip 20 (which is pressed to close and lifted to open the fastener). A saw toothed 22 hinged fastening jaw 24 and a saw toothed 26 stationary jaw 28 which are held together with a hinge pen 30. The free end of the head strap is then folded back to and from the size adjustment loop 32. The loop is terminated by threading the strap's free end thru the loop clevis slot 6 where the end is folded over the loop clevis hinge pin 14 and sew stitched in place completing the adjustable head band assembly. The adjustable tensioning strap 16 is made from a woven rubber and synthetic material that is elastic when stretched.

The strap is 38 inches×1⅛ inches wide× 1/16 inches thick. One end of the strap forms the head band loop 34. The loop is formed by folding 3 inches of the strap's end over sew stitching 38. The opposite end of the tensioning strap is threaded thru the loop clevis 8 then thru the fastener loop 18. The strap is then folded back forming the strap adjustment loop 40. The strap is then thread back thru the loop clevis slot around hinge pin 14 then sew stitched 42 completing the tensioning strap. The apparatus assembly is completed by threading the adjustable head band's free end 4 thru the adjustable tensioning strap's head band loop 34 . FIG. 5 illustrates how the apparatus is to be worn. The adjustable head band loop is adjusted to fit. Then the head band is placed on the head 44 with the tensioning strap centered in the back of the head 46 the length of the tensioning strap is adjusted and the waist band fastener 48 is attached to the waist band 50.

### TECHNICAL FIELD

The method used to aid in correcting head and shoulder dysfunction in the forward position is tensioning. As the head and shoulder tilt too far forward and muscles are too fatigued to counter balance head and shoulders to the appropriate posture position the apparatus go into action.

The tensioning strap is elastic in its nature. It is adjusted with little slack when the head is tilted approximately 15 degrees forward to facilitate unobstructed movement of the head forwardly. Once the head tilts forward further than 15 degrees the tensioning strap starts to engage in putting tension on the muscles that are becoming hyper extended until those muscles exhaust and receive a burst of energy due to increased blood flow as a direct result of muscles going into exhaustion. With an increase of blood flow the previously dormant muscles reenergizes and movement occurs in those muscles, counter balances to move the head and shoulders in the proper position above the trunk of the body as proper body mechanics is supposed to.

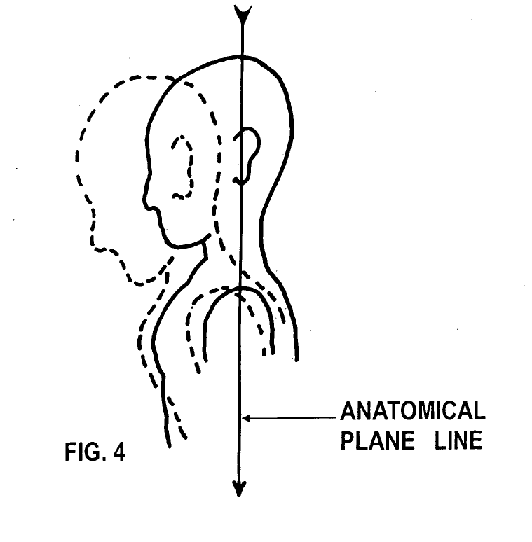

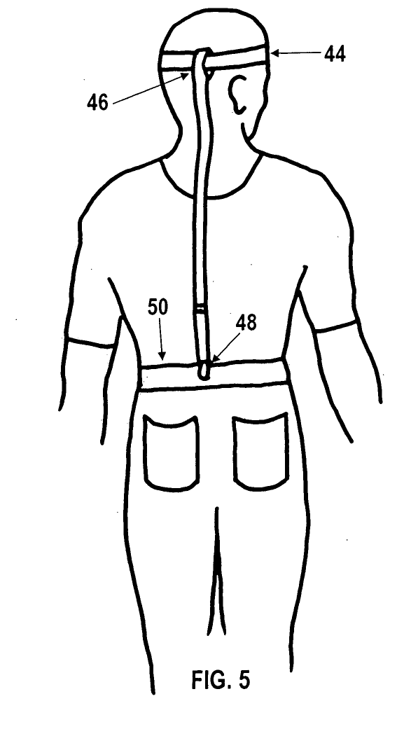

FIG. 4 illustrations show a side view of the head and bust. The dark black solid outlined fig. illustrates the anatomically correct head and shoulder position when one is standing correct (i.e., when the chin is up, head level, ear positioned in line directly over the shoulder with arms resting at the sides.)

The black intermittent or dotted out outline figure illustrates dysfunctional head and should posture in the hyper extended forward position.

We claim:

1. A method for correcting head and shoulder posture comprising placing tension on dysfunction muscles, sensing tension on dysfunction muscles, contracting dysfunctional muscles and thereby bringing head and shoulder into corrective posture and diminishing muscle tension.

2. An apparatus for correcting head and shoulder posture comprising an adjustable head band loop, an adjustable head band strap and an adjustable tension strap for correcting head and shoulder posture and thereby diminishing muscle tension.
